(19) United States
(12) Patent Application Publication
(10) Pub. No.: US 2022/0273798 A1
(43) Pub. Date: Sep. 1, 2022

(54) INERT POLYMER-COATED BIOPHOTONIC SYSTEMS

(71) Applicant: KLOX TECHNOLOGIES INC., Laval (CA)

(72) Inventors: Lise HEBERT, Montreal (CA); Shannon CAMPBELL, Montreal (CA)

(21) Appl. No.: 17/625,503

(22) PCT Filed: Jul. 7, 2020
(86) PCT No.: PCT/CA2020/050942

§ 371 Date: Jan. 7, 2022

Related U.S. Application Data
(60) Provisional application No. 62/871,546, filed on Jul. 8, 2019.

(51) Int. Cl. A61K 41/00; A61K 47/34; A61K 9/70

(52) U.S. Cl. CPC A61K 41/008; A61K 47/34; A61K 9/703

(57) ABSTRACT

The present disclosure generally relates to silicone-coated biophotonic material and to articles comprising same as well as to the potential uses thereof, such as, for example, in wound treatment.

### CROSS-REFERENCE TO RELATED APPLICATION

This application claims the benefit of and priority to U.S. provisional patent application No. 62/871,546, filed on Jul. 8, 2019; the content of all of which is herein incorporated in entirety by reference.

### FIELD OF TECHNOLOGY

The present disclosure generally relates to inert polymer-coated biophotonic materials as well as to their use in biophotonic treatments. In some instances, the present disclosure relates to silicone-coated biophotonic materials as well as to their use in biophotonic treatments.

### BACKGROUND INFORMATION

Biophotonic compositions are now being recognized as having a wide range of applications in the medical, cosmetic and dental fields for use in surgeries, therapies and examinations. For example, biophotonic compositions have been used to treat skin and various tissue disorders as well as to promote wound healing. For these applications, biophotonic therapies have typically been achieved using biophotonic formulations and/or biophotonic compositions comprising light-absorbing molecules capable of absorbing and/or emitting light. These biophotonic formulations and/or compositions have typically been prepared and used as liquids or semi-liquids (e.g., gels, pastes, creams and the like). Due to their liquid and/or semi-liquid texture, some of these biophotonic formulations and/or compositions require a support/surface onto which they can be are applied. Because they tend to spread and/or dilute in contact with fluids, some liquid and semi-liquid biophotonic formulations and/or compositions require multiple applications onto the surface to achieve the desired effect.

Some biophotonic fibers wherein the light-absorbing molecules are integrated into a fiber material have been proposed (e.g., WO 2016/065488, incorporated by reference herein). Such biophotonic fibers alleviate some of the drawbacks observed with the biophotonic formulations and compositions.

Despite the biophotonic fibers known to date, there remains a need in the art for biophotonic materials that provide additional and/or complementary features allowing to expand the scope of biophotonic products that can be created and as well as to expand the scope of therapeutical applications in which these biophotonic products can be used.

### SUMMARY OF DISCLOSURE

According to various aspects, the present disclosure relates to a silicone-coated biophotonic material comprising: at least one biophotonic fiber component coated with silicone, wherein the at least one biophotonic fiber component is photo-stimulated upon exposure to light to emit fluorescence.

Use of the silicone-coated biophotonic material as defined herein for healing of a wound.

Use of the silicone-coated biophotonic material as defined herein in combination with a light source for healing of a wound.

A method for wound healing, the method comprising: applying the silicone-coated biophotonic material as defined herein onto a wound; and illuminating the silicone-coated biophotonic material with actinic light for a time sufficient to achieve photoactivation of the biophotonic fiber component.

According to various aspects, the present disclosure relates to an inert polymer-coated biophotonic material comprising: at least one biophotonic fiber component coated with an inert polymer, wherein the at least one biophotonic fiber component is photo-stimulated upon exposure to light to emit fluorescence. In some implementations, the inert polymer is one or more of hyprophobic, light-transmissible, flexible, non-tearable, and non-heat conductible. In some instances, the inert polymer is a copolymer of tetrafluoroethylene and 2,2-bis(trifluoromethyl)-4,5-difluoro-1,3-dioxole, or is fluorinated ethylene propylene. In some instances, the inert polymer is Teflon™. In some instances, the inert polymer is polytetrafluoroethylene. In some instances, the inert polymer is polyurethane. In some instances, the inert polymer is polydimethylsiloxane.

According to various aspects, the present disclosure relates to the use of the inert polymer-coated biophotonic material as defined herein for healing of a wound.

According to various aspects, the present disclosure relates to the use of the inert polymer-coated biophotonic material as defined herein in combination with a light source for healing of a wound.

According to various aspects, the present disclosure relates to a method for wound healing, the method comprising: applying the inert polymer-coated biophotonic material as defined herein onto a wound; and illuminating the inert polymer-coated biophotonic material with actinic light for a time sufficient to achieve photoactivation of the biophotonic fiber component.

In some implementations of these aspects, the inert polymer material is coated onto the biophotonic material using techniques such as, but not limited to: dip molding, slush molding, rotational molding, casting, spray coating, and the like, which are known in the art.

Other aspects and features of the present technology will become apparent to those ordinarily skilled in the art upon review of the following description of specific embodiments in conjunction with the accompanying drawings.

### DETAILED DESCRIPTION

The present technology is explained in greater detail below. This description is not intended to be a detailed catalog of all the different ways in which the technology may be implemented, or all the features that may be added to the present technology. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which variations and additions do not depart from the present technology. Hence, the following description is intended to illustrate some particular embodiments of the technology, and not to exhaustively specify all permutations, combinations and variations thereof.

As used herein, the singular form “a,” “an” and “the” include plural referents unless the context clearly dictates otherwise. The recitation herein of numerical ranges by endpoints is intended to include all numbers subsumed within that range (e.g., a recitation of 1 to 5 includes 1, 1.5, 2, 2.75, 3, 3.80, 4, 4.32, and 5).

The term “about” is used herein explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. For example, the term “about” in the context of a given value or range refers to a value or range that is within 20%, preferably within 15%, more preferably within 10%, more preferably within 9%, more preferably within 8%, more preferably within 7%, more preferably within 6%, and more preferably within 5% of the given value or range.

The expression “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

The term “biophotonic” as used herein refers to the generation, manipulation, detection and application of photons in a biologically relevant context. As used herein, the expression “biophotonic composition” refers to a light-absorbing-molecules containing composition as described herein that may be illuminated to produce photons for biologically relevant applications. As used herein, the expression “biophotonic regimen” or “biophotonic treatment” or “biophotonic therapy” refers to the use of a combination of a biophotonic composition as defined herein and emitted wavelengths from a light source given at an illumination period of that biophotonic composition.

Terms and expressions “light-absorbing molecule”, “light-capturing molecule”, “photoactivating agent”, “chromophore” and “photoactivator” are used herein interchangeably. A light-absorbing molecule means a molecule or a complex of molecules, which when contacted by light irradiation, is capable of absorbing the light. The light-absorbing molecules readily undergo photoexcitation and in some instances can then transfer its energy to other molecules or emit it as light.

The term “actinic light” as used herein refers to light energy emitted from a specific light source (e.g., lamp, LED, or laser, or variations thereof) and capable of being absorbed by matter (e.g., the light-absorbing molecule defined above). In some embodiments, the actinic light is visible light.

As used herein, the term “treated”, “managed” in expressions such as: “treated tissue”, “managed tissue”, “managed skin”, “treated skin” and “managed area/portion of the skin”, “treated area/portion of the skin”, “managed soft tissue” and “treated soft tissue”, refers to a skin or soft tissue surface or layer(s) onto which a method according to the embodiments of the present technology has been performed.

In some aspects of these embodiments, the expression “biological tissue” refers to any organ and tissue of a living system or organism. Examples of biological tissue include, but are not limited to: brain, the cerebellum, the spinal cord, the nerves, blood, heart, blood vessels, skin, hair, fat, nails, bones, cartilage, ligaments, tendons, ovaries, fallopian tubes, uterus, vagina, bone, mammary glands, testes, vas deferens, seminal vesicles, prostate, salivary glands, esophagus, stomach, liver, gallbladder, pancreas, intestines, rectum, anus, kidneys, ureters, bladder, urethra, the pharynx, larynx, bronchi, diaphragm, hypothalamus, pituitary gland, pineal body or pineal gland, thyroid, parathyroid, adrenals (e.g., adrenal glands), lymph nodes and vessels, skeletal muscles, smooth muscles, cardiac muscle, peripheral nervous system, ears, eyes, nose, gums, nails, scalp, and the like.

As used herein, the term “fiber” relates to a string or a thread or a filament used as a component of composite materials. Fibers may be used in the manufacture of other materials such as for example, but not limited to, yarns and fabrics.

As used herein, the expression “woven” refers to a material (e.g., fabric) that is formed by weaving. As used herein, the expression “non-woven” refers to a material (e.g., fabric) that is made from staple fibers (short) and long fibers (continuous long), bonded together by chemical, mechanical, heat or solvent treatment. The expression “non-woven” may be used herein to denote a material which is neither woven nor knitted (e.g., a felt). As used herein, a “felt” is a textile that is produced by matting, condensing and pressing fibers together. As used herein, the term “carding” refers to a mechanical process that disentangles, cleans and intermixes fibres to produce a continuous web or sliver suitable for subsequent processing. This is achieved by passing the fibers between differentially moving surfaces covered with card clothing. It breaks up locks and unorganised clumps of fiber and then aligns the individual fibers to be parallel with each other.

As used herein the term “wound” refers to an injury in which skin is torn, cut, or punctured (i.e., an open wound), or where blunt force trauma causes a contusion (i.e., closed wound), or sutured wound. Open wounds can be classified according to the object that caused the wound: Incisions or incised wounds are caused by a clean, sharp-edged object such as a knife, razor, or glass splinter. Lacerations are irregular tear-like wounds caused by some blunt trauma. Lacerations and incisions may appear linear (regular) or stellate (irregular). The term laceration is commonly misused in reference to incisions. Abrasions (grazes) are superficial wounds in which the topmost layer of the skin (the epidermis) is scraped off. Abrasions are often caused by a sliding fall onto a rough surface. Avulsions are injuries in which a body structure is forcibly detached from its normal point of insertion. A type of amputation where the extremity is pulled off rather than cut off. Puncture wounds are caused by an object puncturing the skin, such as a splinter, nail or needle. Penetration wounds are caused by an object such as a knife entering and coming out from the skin. Gunshot wounds are caused by a bullet or similar projectile driving into or through the body. There may be two wounds, one at the site of entry and one at the site of exit, generally referred to as a “through-and-through”. Wounds suffered from blast injuries. Closed wounds include: Hematomas (or blood tumor) which are caused by damage to a blood vessel that in turn causes blood to collect under the skin. Hematomas that originate from internal blood vessel pathology are petechiae, purpura, and ecchymosis. The different classifications are based on size. Hematomas that originate from an external source of trauma are contusions, also commonly called bruises. Crush injury are caused by a great or extreme amount of force applied over a long period of time. According to level of contamination, a wound can be classified as: a clean wound which is made under sterile conditions where there are no organisms present and the skin is likely to heal without complications. Contaminated wounds are usually resulting from accidental injury; there are pathogenic organisms and foreign bodies in the wound. Infected wounds are the wound with pathogenic organisms present and multiplying, exhibiting clinical signs of infection (yellow appearance, soreness, redness, oozing pus). Colonized wound is a chronic situation, containing pathogenic organisms, difficult to heal (i.e., bedsore). Wounds that are said to be acute are typically categorized as two main types: traumatic wounds and surgical wounds. Wounds that are said to be chronic are wounds that do not heal in an orderly set of stages and in a predictable amount of time the way most wounds do; wounds that do not heal within three months are often considered chronic. Chronic wounds seem to be detained in one or more of the phases of wound healing.

Wound dressings can be used to cover wounds in an effort to assist in the wound healing process. In general, wound dressings can be classified as passive or active types, depending on their roles in wound healing. Passive wound dressings refer to the dressings which only provide a cover for the wound at the basic level, whereas wound dressings are those facilitating the management of the wound and promoting wound healing. An ideal wound dressing will possess certain characteristics in order to help with the wound healing process. Examples of desired characteristics include, the ability to retain and absorb moisture, allowing good permeation of gas, particularly for the supply of oxygen from the ambient air to the covered wound area and for removal of excess carbon dioxide from the wound area to the ambient air, as well as for control of bacterial growth. Biophotonic compositions have also been proposed to assist wound dressing in the promotion of healing of wounds such as chronic wounds (see, in particular, WO 2015/000058, incorporated herein, in its entirety, by reference).

The biophotonic fibers of the present disclosure comprise light-absorbing molecules that are photoactivatable or photostimulated by photoactivation or photostimulation of the biophotonic fibers. In some instances, the light-absorbing molecules are present on the surface of the biophotonic fibers (e.g., the biophotonic fibers are coated or sprayed with the light-absorbing molecules or the fibers are dipped into a composition or a formulation comprising the light-absorbing molecules). In other instances, the light-absorbing molecules are incorporated into the materials making the biophotonic fibers (e.g., the light-absorbing molecules are mixed/compounded with the materials making the biophotonic fibers). In some other implementations, the light-absorbing molecules are present both on the surface of the biophotonic fibers and incorporated/compounded into the materials making the biophotonic fibers.

In some instances, the biophotonic fibers are, but not limited to, synthetic fibers, natural fibers, and textile fibers. For example, synthetic fibers may be made from a polymer or a combination of different polymers. In some instances, the polymer is a thermoplastic polymer. In some implementations, the biophotonic fibers of the present disclosure are as described in WO2016/065488, incorporated herein in its entirety by reference.

In some instances, the polymer is acrylic, acrylonitrile butadiene styrene (ABS), polybenzimidazole (PBI), polycarbonate, polyether sulfone (PES), polyetherether ketone (PEEK), polyetherimide (PEI), polyethylene (PE), polyphenylene oxide (PPO), polyphenylene sulfide (PPS), polypropylene (PP), polystyrene, polyvinyl chloride (PVC), teflon, polybutylene, polyethylene terephthalate (PET), polybutylene terephthalate (PBT), nylon, polylactic acid (PLA), polymethyl methacrylate polyester, polyurethane, rayons, poly(methyl methacrylate) (PMMA), or from any mixture thereof.

In some other instances, the biophotonic fibers may be made from glycolic acid, copolymer lactide/glycolide, polyester polymer, copolymer polyglycolic acid/trimethylene carbonate, natural protein fiber, cellulose fiber, polyamide polymer, polymer of polypropylene, polymer of polyethylene, nylon, polymer of polylactic acid, polymer of polybutylene terephthalate, polyester, copolymer polyglycol, polybutylene, polymer of poly methyl methacrylate, or from any mixture thereof.

In some implementations, the biophotonic fibers of the present disclosure may be coextruded fibers that have two distinct polymers forming the biophotonic fibers, usually as a core-sheath or side-by-side.

In some implementations, the diameter of the biophotonic fibers (taken individually, monofilament) varies between about 15 microns and about 500 microns, between about 25 microns and about 500 microns, between about 50 microns and 400 microns, between about 50 microns and about 300 microns, preferably between about 50 microns and about 250 microns, preferably between about 75 microns and about 300 microns, and most preferably between about 75 microns and about 250 microns. In some specific implementations, the diameter of the biophotonic fibers defined herein is about 15 microns, about 20 microns, about 25 microns, about 50 microns, about 75 microns, about 100 microns, about 125 microns, about 150 microns, about 175 microns, about 200 microns, about 225 microns, about 250 microns, about 275 microns, about 300 microns, about 325 microns, about 350 microns, about 375 microns, about 400 microns, about 425 microns, about 450 microns, about 475 microns, about 500 microns. In some instances, the diameter of the biophotonic fibers defined herein (taken individually) is about 31 microns.

In some implementations, the biophotonic fibers have a linear mass density of between about 300 and about 480 Deniers, between about 410 and about 470 Deniers, between about 420 and about 460 Deniers, between about 420 and about 450 Deniers, or about 428 Deniers. As used herein, the term “Denier” refers to a unit of measure for the linear mass density of fibers, is defined as the mass in grams per 9000 meters.

In some embodiments, the biophotonic fibers of the present disclosure are prepared by an extrusion process wherein polymer pellets are melted and extruded and then pulled into a fiber while still hot. The fibers were dipped in Lurol Oil™/Water solution (10%). The fibers are then spun onto a bobbin for storage and ease of use. In some instances, the biophotonic fibers of the present disclosure are prepared using a TEM co-rotating twin screw extruder.

In some implementations, the light-absorbing molecule is a chemical compound which, when exposed to the light is photoexcited and can then transfer its energy to other molecules or emit it as light, such as for example fluorescence. For example, in some instances, the light-absorbing molecule when photoexcited by the light may transfer its energy to enhance or accelerate light dispersion. Examples of light-absorbing molecules include, but are not limited to, fluorescent compounds (or stains) (also known as “fluorochromes” or “fluorophores” or “chromophores”). Other dye groups or dyes (biological and histological dyes, food colorings, carotenoids, and other dyes) can also be used. Suitable light-absorbing molecule can be those that are Generally Regarded As Safe (GRAS).

In certain implementations, the biophotonic fibers of the present disclosure comprise a first light-absorbing molecule. In some implementations, the first light-absorbing molecule absorbs at a wavelength in the range of the visible spectrum, such as at a wavelength of about 380 nm to about 1000 nm, about 380 nm to about 800 nm, about 380 nm to about 700 nm, about 400 nm to about 800 nm, or about 380 nm to about 600 nm. In other embodiments, the first light-absorbing molecule absorbs at a wavelength of about 200 nm to about 1000 nm, about 200 nm to about 800 nm, of about 200 nm to about 700 nm, of about 200 nm to about 600 nm or of about 200 nm to about 500 nm. In one embodiment, the first light-absorbing molecule absorbs at a wavelength of about 200 nm to about 600 nm. In some embodiments, the first light-absorbing molecule absorbs light at a wavelength of about 200 nm to about 300 nm, of about 250 nm to about 350 nm, of about 300 nm to about 400 nm, of about 350 nm to about 450 nm, of about 400 nm to about 500 nm, of about 450 nm to about 650 nm, of about 600 nm to about 700 nm, of about 650 nm to about 750 nm or of about 700 nm to about 800 nm. In some implementations, the light-absorbing molecule emits light within the range of about 400 nm and about 800 nm. In certain embodiments, the fluence delivered to the treatment areas may be between about 0.001 to about 60 J/cm², about 4 to about 60 J/cm², about 10 to about 60 J/cm², about 10 to about 50 J/cm², about 10 to about 40 J/cm², about 10 to about 30 J/cm², about 20 to about 40 J/cm², about 15 J/cm² to 25 J/cm², or about 10 to about 20 J/cm². In some embodiments, the fluence delivered to the treatment areas after 5 minutes of illumination is between about 33 J/cm² and about 45 J/cm², or between about 55 J/cm² and about 129 J/cm².

The biophotonic fibers disclosed herein may include at least one additional light-absorbing molecule. Combining light-absorbing molecules may increase photo-absorption by the combined light-absorbing molecules and enhance absorption and photo-biomodulation selectivity. Thus, in certain embodiments, the biophotonic fibers of the disclosure include more than one light-absorbing molecule.

In other implementations wherein the biophotonic fibers have the light-absorbing molecule on their surface (i.e., the surface of the fibers that is in contact with the surrounding environment of the fiber), such biophotonic fibers may be prepared by being sprayed with a light-absorbing molecule composition comprising one or more light-absorbing molecules and a carrier material.

In some specific examples, the light-absorbing molecule composition has a consistency that allows the fibers to be dipped into the composition. In some specific examples, the light-absorbing molecule composition is in a liquid or semi-liquid form. The carrier material may be any liquid or semi liquid material that is compatible with the light-absorbing molecule that is any material that does not affect the photoactive properties of the light-absorbing molecule, such as, for example, water. In some other specific examples, the light-absorbing molecule composition has a consistency that allows the light-absorbing molecule composition to be sprayed onto the fibers.

In the implementations wherein the biophotonic fibers have the light-absorbing molecule incorporated into the fibers, the biophotonic fibers are prepared by incorporating the light-absorbing molecule into the fiber composition. In some examples, the biophotonic fibers are prepared by extrusion. In some specific implementations, the biophotonic fibers are prepared by a process which uses spinning. The spinning may be wet, dry, dry jet-wet, melt, or gel. The polymer being spun may be converted into a fluid state. If the polymer is a thermoplastic then it may be melted, otherwise it may be dissolved in a solvent or may be chemically treated to form soluble or thermoplastic derivatives. The molten polymer is then forced through the spinneret, and then it cools to a rubbery state, and then a solidified state. If a polymer solution is used, then the solvent is removed after being forced through the spinneret. A composition of the light-absorbing molecule may be added to the polymer in the fluid state or to the melted polymer or to the polymer dissolved into a solvent. Melt spinning may be used for polymers that can be melted. The polymer having the light-absorbing molecules dispersed therein solidifies by cooling after being extruded from the spinneret.

The concentration of the light-absorbing molecule to be used may be selected based on the desired intensity and duration of the photoactivity to be emitted from the biophotonic fibers, and on the desired phototherapeutic, medical or cosmetic effect. For example, some dyes such as xanthene dyes reach a ‘saturation concentration’ after which further increases in concentration do not provide substantially higher emitted fluorescence. Further increasing the light-absorbing molecule concentration above the saturation concentration can reduce the amount of activating light passing through the biophotonic fibers. Therefore, if more fluorescence is required for a certain application than activating light, a high concentration of light-absorbing molecule can be used. However, if a balance is required between the emitted fluorescence and the activating light, a concentration close to or lower than the saturation concentration can be chosen.

Suitable light-absorbing molecule that may be used in the biophotonic fibers of the present disclosure include, but are not limited to the following: chlorophyll dyes, xanthene derivatives, methylene blue dyes and azo dyes. Examples of xanthene derivatives include, but are not limited to: eosin, eosin B (4′,5′-dibromo,2′,7′-dinitr-o-fluorescein, dianion); eosin Y; eosin Y (2′,4′,5′,7′-tetrabromo-fluorescein, dianion); eosin (2′,4′,5′,7′-tetrabromo-fluorescein, dianion); eosin (2′,4′,5′,7′-tetrabromo-fluorescein, dianion) methyl ester; eosin (2′,4′,5′,7′-tetrabromo-fluorescein, monoanion) p-isopropylbenzyl ester; eosin derivative (2′,7′-dibromo-fluorescein, dianion); eosin derivative (4′,5′-dibromo-fluorescein, dianion); eosin derivative (2′,7′-dichloro-fluorescein, dianion); eosin derivative (4′,5′-dichloro-fluorescein, dianion); eosin derivative (2′,7′-diiodo-fluorescein, dianion); eosin derivative (4′,5′-diiodo-fluorescein, dianion); eosin derivative (tribromo-fluorescein, dianion); eosin derivative (2′,4′,5′,7′-tetrachlor-o-fluorescein, dianion); eosin dicetylpyridinium chloride ion pair; erythrosin B (2′,4′,5′,7′-tetraiodo-fluorescein, dianion); erythrosin; erythrosin dianion; erythiosin B; fluorescein; fluorescein dianion; phloxin B (2′,4′,5′,7′-tetrabromo-3,4,5,6-tetrachloro-fluorescein, dianion); phloxin B (tetrachloro-tetrabromo-fluorescein); phloxine B; rose bengal (3,4,5,6-tetrachloro-2′,4′,5′,7′-tetraiodofluorescein, dianion); pyronin G, pyronin J, pyronin Y; Rhodamine dyes such as rhodamines that include, but are not limited to, 4,5-dibromo-rhodamine methyl ester; 4,5-dibromo-rhodamine n-butyl ester; rhodamine 101 methyl ester; rhodamine 123; rhodamine 6G; rhodamine 6G hexyl ester; tetrabromo-rhodamine 123; and tetramethyl-rhodamine ethyl ester.

In some embodiments, the light-absorbing molecule is an endogeneous molecules such as, but not limited to, vitamins. Examples of vitamins that may act as endogenous light-absorbing molecules include, vitamin B. In some instances, the endogenous light-absorbing molecule is vitamin B12. In some instances, the endogenous light-absorbing molecule is 7,8-Dimethyl-10-[(2S,3S,4R)-2,3,4,5-tetrahydroxypentyl]benzo[g]pteridine-2,4-dione.

In certain embodiments, the biophotonic fibers of the present disclosure may include any of the light-absorbing molecules listed above, or a combination thereof, so as to provide a synergistic biophotonic effect. For example, the following synergistic combinations of light-absorbing molecules may be used: Eosin Y and Fluorescein; Fluorescein and Rose Bengal; Erythrosine in combination with Eosin Y, Rose Bengal or Fluorescein; Phloxine B in combination with one or more of Eosin Y, Rose Bengal, Fluorescein and Erythrosine; Eosin Y, Fluorescein and Rose Bengal.

In some examples, the light-absorbing molecule is present in the light-absorbing molecule composition at a concentration of about 100 g/L, about 50 g/L, about 10 g/L, about 5 g/L, about 1 g/L or about 0.1 g/L of the total volume. Preferably, the light-absorbing molecule is present in the light-absorbing molecule composition at a concentration of between about 10 g/L and about 100 g/L. In some instances, the light-absorbing molecule is present in the light-absorbing molecule composition at a concentration that is lower than 0.1 g/L, for example, the light-absorbing molecule is present in the light-absorbing molecule composition at a concentration in the milligram/L or in the microgram/L range.

In some embodiments, the biophotonic fibers of the present disclosure comprise a lubricant. In some instances, the lubricant is coated onto the biophotonic fibers of the present disclosure. In some instances, the lubricant is treatment oil, such as but not limited to Lurol Oil™.

In some implementations, there is less than about 15% leaching of the light-absorbing molecule out of the biophotonic fibers of the present disclosure, more preferably less than 10%, more preferably less than 5%, more preferably less than 4%, more preferably less than 3%, more preferably less than 2%, more preferably less than 1%, or even more preferably substantially no leaching of the light-absorbing molecule out of the biophotonic fibers. Leaching of the light-absorbing molecule out of the biophotonic fibers of the present disclosure may be assessed by placing 0.1 g of the biophotonic fibers in 10 ml of water for 1 day and by then measuring the amount of light-absorbing molecule in the water.

In some implementations, the biophotonic fibers as defined herein may be woven into a fabric material resulting in a biophotonic fabric comprising a plurality of biophotonic fibers. In some implementations, the biophotonic fabric comprising the biophotonic fibers exhibits substantially no leaching of the light-absorbing molecule. In some implementations, the biophotonic fibers as defined herein may be bonded together by entangling the fibers mechanically, thermally or chemically to create a non-woven material. In some examples, the biophotonic woven or non-woven material may be used in the fabrication of an article of manufacture such as, but not limited to, a garment, an article of clothing, a wound dressing, a towel, bedding, and the like. In some implementation the garment may be a shirt, pants, glove, mask, socks, or the like.

In some implementations, the biophotonic fibers as defined herein may be woven into a mesh resulting in a biophotonic mesh. As used herein, the expression “biophotonic mesh” refers to a loosely woven sheet of biophotonic fibers.

In the implementations wherein the light-absorbing molecules are compounded with the polymer of the fibers, the compounded polymer or the mesh made from such fibers is also photoactivatable. Whereas in the implementations wherein the light-absorbing molecules are not compounded with the polymer of the fibers, the fabric or the mesh made from such fibers may be coated or dipped or sprayed with a light-absorbing molecule composition to render the fabric photoactivatable.

In some other examples, the biophotonic fibers may be a non-woven biophotonic fabric or biophotonic mesh. Such biophotonic fabric and biophotonic mesh may be produced by depositing extruded, spun filaments onto a collecting belt in a uniform random manner followed by bonding the fibers. The fibers may be separated during the web laying process by air jets or electrostatic charges. The collecting surface is usually perforated to prevent the air stream from deflecting and carrying the fibers in an uncontrolled manner. Bonding imparts strength and integrity to the web by applying heated rolls or hot needles to partially melt the polymer and fuse the fibers together. In general, high molecular weight and broad molecular weight distribution polymers such as, but not limited to, polypropylene, polyester, polyethylene, polyethylene terephthalate, nylon, polyurethane, and rayons may be used in the manufacture of spunbound fabrics. In some instances, the biophotonic fabrics or biophotonic mesh may be composed of a mixture of polymers. A lower melting polymer can function as the binder which may be a separate fiber interspersed with higher melting fibers, or two polymers may be combined into a single fiber type. In the latter case the so-called bi-component fibers possess a lower melting component, which acts as a sheath covering over a higher melting core. Bicomponent fibers may also spun by extrusion of two adjacent polymers.

In some instances, spunbonding may combine fiber spinning with web formation by placing the bonding device in line with spinning. In some arrangements the web may be bonded in a separate step. The spinning process may be similar to the production of continuous filament yarns and may utilize similar extruder conditions for a given polymer. Fibers are formed as the molten polymer exits the spinnerets and is quenched by cool air. The objective of the process is to produce a wide web and, therefore, many spinnerets are placed side by side to generate sufficient fibers across the total width.

Before deposition on a moving belt or screen, the output of a spinneret usually includes a plurality of individual filaments which must be attenuated to orient molecular chains within the fibers to increase fiber strength and decrease extensibility. This is accomplished by rapidly stretching the plastic fibers immediately after exiting the spinneret. In practice the fibers are accelerated either mechanically or pneumatically. The web is formed by the pneumatic deposition of the filament bundles onto the moving belt. A pneumatic gun uses high-pressure air to move the filaments through a constricted area of lower pressure, but higher velocity as in a venturi tube. In order for the web to achieve maximum uniformity and cover, individual filaments are separated before reaching the belt. This is accomplished by inducing an electrostatic charge onto the bundle while under tension and before deposition. The charge may be induced triboelectrically or by applying a high voltage charge. The belt is usually made of an electrically grounded conductive wire. Upon deposition, the belt discharges the filaments. Webs produced by spinning linearly arranged filaments through a so-called slot die eliminating the need for such bundle separating devices.

Many methods can be used to bond the fibers in the spun web. These include mechanical needling, thermal bonding, and chemical bonding. The last two may bond large regions (area bonding) or small regions (point bonding) of the web by fusion or adhesion of fibers. Point bonding results in the fusion of fibers at points, with fibers between the point bonds remaining relatively free. Other methods used with staple fiber webs, but not routinely with continuous filament webs include stitch bonding, ultrasonic fusing, and hydraulic entanglement.

In some embodiments, the biophotonic fabrics and the biophotonic mesh of the present technology have interstices present between the biophotonic fibers making up the biophotonic fabrics or the biophotonic mesh.

The biophotonic fibers and biophotonic mesh of the present disclosure comprises a silicone coating. In certain embodiments, the silicone coating of the present disclosure can be prepared by using commercial kits such as MED-4011, MED-6015, and/or MED-6350 provided by NuSir. The kit consists in two-part liquid components, the base (part A) and the curing agent or catalyst (part B), both based on polydimethylsiloxane. When mixed at a ratio of 10(A)/1(B) or 1(A)/1(B) the mixture cures to a flexible and transparent elastomer. MED-6015 (“low consistency silicone”) is a silicone elastomer comprising a polydimethyl siloxane and organically-modified silica. The low consistency silicone is prepared by combining a base (Part A) with a curing agent (Part B). The base contains about >60 wt % dimethylvinyl-terminated dimethyl siloxane, about 30 to 60 wt % dimethylvinylated and trimethylated silica and about 1 to 5 wt % tetra(trimethylsiloxy) silane. The curing agent contains about 40 to 70 wt % dimethyl, methylhydrogen siloxane, about 15 to 40 wt % dimethylvinyl-terminated dimethyl siloxane, about 10 to 30 wt % dimethylvinylated and trimethylated silica and about 1 to 5 wt % tetramethyl tetravinyl cyclotetrasiloxane.

In another embodiment, the silicone coating can be prepared by using the MED-6360 (“soft adhesive silicone”) kit, which allows the preparation of a soft and sticky gel, when the two parts A and B are mixed at the ratio 1(A)/1(B). Parts A and B of the kit contain about 85 to 100 wt % dimethylvinyl-terminated dimethyl siloxane and about 1 to 5 wt % dimethyl, methylhydrogen siloxane.

In other embodiments, the silicone coating may be prepared in a manner to provide for tunable flexibility were desired, for example a silicone-based biophotonic membrane having tunable flexibility. One means of generating a tunable biophotonic silicone membrane of the present disclosure is by combining different ratios of commercially available PDMS such as MED-4011, MED-6015, and/or MED-6350. In some embodiments the silicone phase comprises MED-6360 in the amount of 5-100 wt % of the silicone phase. In certain embodiments of the present disclosure the MED-6350 is present in an amount of about 5-10 wt %, 10-15 wt %, 15-20 wt %, 20-25 wt %, 25-30 wt %, 30-35 wt %, 35-40 wt %, 40-45 wt %, 45-50 wt %, 50-55 wt %, 55-60 wt %, 60-65 wt % 65-70 wt %, 70-75 wt %, 75-80 wt %, 80-85 wt %, 85-90 wt %, 90-95 wt % or 95-100 wt % of the silicone phase. In certain embodiments of the present disclosure, the silicone phase comprises MED-6015. In certain other embodiments of the present disclosure, the MED-6015 is present in an amount of about 5-10 wt %, 10-15 wt %, 15-20 wt %, 20-25 wt %, 25-30 wt %, 30-35 wt %, 35-40 wt %, 40-45 wt %, 45-50 wt %, 50-55 wt %, 55-60 wt %, 60-65 wt % 65-70 wt %, 70-75 wt %, 75-80 wt %, 80-85 wt %, 85-90 wt %, 90-95 wt % or 95-100 wt % of the silicone phase. In certain other embodiments of the present disclosure, the MED-4011 is present in an amount of about 5-10 wt %, 10-15 wt %, 15-20 wt %, 20-25 wt %, 25-30 wt %, 30-35 wt %, 35-40 wt %, 40-45 wt %, 45-50 wt %, 50-55 wt %, 55-60 wt %, 60-65 wt % 65-70 wt %, 70-75 wt %, 75-80 wt %, 80-85 wt %, 85-90 wt %, 90-95 wt % or 95-100 wt % of the silicone phase.

In some embodiments, the silicone coating is applied to the biophotonic fibers or to the biophotonic mesh of the present disclosure by immersing or dipping the biophotonic fibers or the biophotonic mesh into a silicone melt. In some other embodiments, the silicone coating is applied to the biophotonic fibers or to the biophotonic mesh of the present disclosure by spraying the biophotonic fibers or the biophotonic mesh with a silicone melt.

In certain embodiments, silicone coating has a thickness in a range of about 10 μm to about 100 μm. In some embodiments, the outer coating has a thickness in a range of about 10 μm to about 75 μm, about 10 μm to about 50 μm, about 10 μm to about 25 μm, or about 20 μm.

In some embodiments, the silicone-coating biophotonic materials of the present disclosure may have therapeutic and/or cosmetic and/or medical benefits. In some implementations of these embodiments, the silicone-coating biophotonic material may be used to promote the prevention and/or treatment of a tissue or an organ and/or to treat a tissue or an organ of a subject in need of phototherapy. In some instances, the silicone-coating biophotonic material may be used to promote wound healing. In this case, the silicone-coating biophotonic material may be applied at wound site as deemed necessary by the physician or other health care providers, or home patient caregivers. In certain embodiments, the silicone-coating biophotonic material may be used following wound closure to optimize scar revision. In this case, the silicone-coating biophotonic material may be applied at regular intervals such as once a week, or at an interval deemed appropriate by the physician or other health care providers. Wounds that may be treated by the silicone-coated biophotonic material of the present disclosure include, for example, injuries to the skin and subcutaneous tissue initiated in different ways (e.g., surgical site infection, pressure ulcers from extended bed rest, colonized or infected wounds, wounds induced by trauma or surgery, burns (including early stages of burns), ulcers linked to diabetes or venous insufficiency) and with varying characteristics. In certain embodiments, the present disclosure provides silicone-coated biophotonic material for treating and/or promoting the healing of, for example, burns, burns related to blast injuries, burns related to chemical and/or radiation burns (suffered during combat injuries), incisions, excisions, lesions, lacerations, abrasions, puncture or penetrating wounds, surgical wounds, contusions, hematomas, crushing injuries, amputations, sores and ulcers.

In certain embodiments, the silicone-coated biophotonic materials of the present disclosure are used in conjunction with systemic or topical antibiotic treatment (such as, for examples: tetracycline, erythromycin, minocycline, doxycycline). In some implementations, the article of manufacture being composed of the silicone-coated biophotonic materials of the present disclosure may be able to control bacterial growth, for example when used in the treatment of a wound to minimize undesirable clinical outcomes associated with bacterial colonized wounds.

In some embodiments, the biophotonic fibers and fabrics of the present disclosure may be used in a method for effecting phototherapy on a subject, such as on a tissue (e.g., wounded tissue) of the subject. Such method comprises the step of applying an silicone-coated biophotonic material as defined herein onto the subject or onto the tissue in need of phototherapy and the step of illuminating the silicone-coated biophotonic material with light having a wavelength that overlaps partially, or in full, with an absorption spectrum of the light-absorbing molecule.

In the methods of the present disclosure, any source of actinic light can be used. Any type of halogen, LED or plasma arc lamp, or laser may be suitable. The primary characteristic of suitable sources of actinic light will be that they emit light in a wavelength (or wavelengths) appropriate for illumination of the one or more light-absorbing molecule present in the silicone-coated biophotonic materials. In one embodiment, an argon laser is used. In another embodiment, a potassium-titanyl phosphate (KTP) laser (e.g. a GreenLight™ laser) is used. In yet another embodiment, a LED lamp such as a photocuring device is the source of the actinic light. In yet another embodiment, the source of the actinic light is a source of light having a wavelength between about 200 nm to 800 nm. In another embodiment, the source of the actinic light is a source of visible light having a wavelength between about 400 nm and 600 nm. In another embodiment, the source of the actinic light is a source of visible light having a wavelength between about 400 nm and 700 nm.

In yet another embodiment, the source of the actinic light is blue light. In yet another embodiment, the source of the actinic light is red light. In yet another embodiment, the source of the actinic light is green light. Furthermore, the source of actinic light should have a suitable power density. Suitable power densities for non-collimated light sources (LED, halogen or plasma lamps) are in the range from about 0.1 mW/cm² to about 200 mW/cm². Suitable power densities for laser light sources are in the range from about 0.5 mW/cm² to about 0.8 mW/cm².

In some implementations, the light has an energy at the subject's skin surface of between about 0.001 mW/cm² and about 500 mW/cm², or 0.1-300 mW/cm², or 0.1-200 mW/cm², wherein the energy applied depends at least on the condition being treated, the wavelength of the light, the distance of the tissue from the light source and the thickness of the silicone-coated biophotonic materials. In certain embodiments, the light at the subject's tissue is between about 1-40 mW/cm², or between about 20-60 mW/cm², or between about 40-80 mW/cm², or between about 60-100 mW/cm², or between about 80-120 mW/cm², or between about 100-140 mW/cm², or between about 30-180 mW/cm², or between about 120-160 mW/cm², or between about 140-180 mW/cm², or between about 160-200 mW/cm², or between about 110-240 mW/cm², or between about 110-150 mW/cm², or between about 190-240 mW/cm².

Photoactivation of the light-absorbing molecules may take place almost immediately on illumination (femto- or pico seconds). A prolonged exposure period may be beneficial to exploit the synergistic effects of the absorbed, reflected and reemitted light of the biophotonic fibers and fabrics of the present disclosure and its interaction with the tissue being treated. In one embodiment, the time of exposure of silicone-coated biophotonic materials to actinic light is a period between 0.01 minutes and 90 minutes. In another embodiment, the time of exposure of the silicone-coated biophotonic materials to actinic light is a period between 1 minute and 5 minutes. In some other embodiments, the silicone-coated biophotonic materials are illuminated for a period between 1 minute and 3 minutes. In certain embodiments, light is applied for a period of about 1-30 seconds, about 15-45 seconds, about 30-60 seconds, about 0.75-1.5 minutes, about 1-2 minutes, about 1.5-2.5 minutes, about 2-3 minutes, about 2.5-3.5 minutes, about 3-4 minutes, about 3.5-4.5 minutes, about 4-5 minutes, about 5-10 minutes, about 10-15 minutes, about 15-20 minutes, or about 20-30 minutes. The treatment time may range up to about 90 minutes, about 80 minutes, about 70 minutes, about 60 minutes, about 50 minutes, about 40 minutes or about 30 minutes. It will be appreciated that the treatment time can be adjusted in order to maintain a dosage by adjusting the rate of fluence delivered to a treatment area. For example, the delivered fluence may be about 4 to about 60 J/cm², 4 to about 90 J/cm², 10 to about 90 J/cm², about 10 to about 60 J/cm², about 10 to about 50 J/cm², about 10 to about 40 J/cm², about 10 to about 30 J/cm², about 20 to about 40 J/cm², about 15 J/cm² to J/cm², or about 10 to about 20 J/cm², or about 0.001 J/cm² to about 1 J/cm².

In certain embodiments, the silicone-coated biophotonic materials may be re-illuminated at certain intervals. In yet another embodiment, the source of actinic light is in continuous motion over the treated area for the appropriate time of exposure. In yet another embodiment, the silicone-coated biophotonic materials may be illuminated until the silicone-coated biophotonic materials is at least partially photobleached or fully photobleached.

In certain embodiments, the light-absorbing molecules in the silicone-coated biophotonic materials can be photoexcited by ambient light including from the sun and overhead lighting. In certain embodiments, the light-absorbing molecules can be photoactivated by light in the visible range of the electromagnetic spectrum. The light can be emitted by any light source such as sunlight, light bulb, an LED device, electronic display screens such as on a television, computer, telephone, mobile device, flashlights on mobile devices. In the methods of the present disclosure, any source of light can be used. For example, a combination of ambient light and direct sunlight or direct artificial light may be used. Ambient light can include overhead lighting such as LED bulbs, fluorescent bulbs, and indirect sunlight.

In the methods of the present disclosure, the silicone-coated biophotonic materials may be removed from the tissue following application of light. In other embodiments, the silicone-coated biophotonic materials may be left on the tissue for an extended period of time.

In certain instances, the silicone-coated biophotonic material of the present disclosure may be used in the manufacture of articles such as; medical devices (e.g., wound dressing or the like).

Identification of equivalent compositions, methods and kits are well within the skill of the ordinary practitioner and would require no more than routine experimentation, in light of the teachings of the present disclosure. Practice of the disclosure will be still more fully understood from the following examples, which are presented herein for illustration only and should not be construed as limiting the disclosure in any way.

### EXAMPLES

The examples below are given so as to illustrate the practice of various embodiments of the present technology. They are not intended to limit or define the entire scope of this technology. It should be appreciated that the technology is not limited to the particular embodiments described and illustrated herein but includes all modifications and variations falling within the scope of the disclosure as defined in the appended embodiments.

### Example 1: Fluorescence Emission Properties of a Silicone-Coated Biophotonic Mesh

Light-absorbing molecules were incorporated into fibers made of nylon. The compounding involved taking a nylon melt and adding the light-absorbing molecules in their solid form directly to the nylon melt, and then allowing the melt to cool. This process allowed the light-absorbing molecules to be integrated into the nylon fibers. The light-absorbing molecule to nylon ratio was selected so as to be dependent on the light-absorbing molecules used, for example: for Eosin Y, a 1% w/w ratio (in water) was used was used for the master chromophore batch. Eosin Y or fluorescein or a combination of Eosin Y and fluorescein were used as light-absorbing molecules.

Biophotonic meshes were prepared by knitting biophotonic fibers so as to make a 2 mm thick mesh with a width of 22 cm (2 mm mesh).

The biophotonic mesh was coated with silicone (Nusil® MED 6360) by spraying the silicone on the biophotonic mesh to create a silicone coating having a thickness of 20 microns. The silicone-coated biophotonic meshes were assessed for their ability to emit fluorescence following illumination for 5 minutes at 5 cm using a KT-L™ Lamp. The results are presented in Table 1 for the 2 mm thick mesh.

TABLE 1
Fluorescence emission of a light-stimulated inert
polymer-coated biophotonic woven mesh (2 mm)

| | mW/cm² at 5 cm | | | |
|---|---|---|---|---|
| | 0 min | 5 min | J/cm² | % |
| Lamp (400-518 nm) | 40.49 | 40.24 | 12.13 | 85.0 |
| Fluoresc. (519-760 nm) | 7.76 | 6.25 | 2.13 | 14.9 |
| TOTAL (400-760 nm) | 48.26 | 46.48 | 14.26 | 99.9 |
| % Fluorescence | 16.1 | 13.4 | 0.15 | 14.9 |
| Purple (400-450 nm) | 20.04 | 18.72 | 5.83 | 40.8 |
| Blue (450-500 nm) | 20.39 | 21.43 | 6.28 | 44.0 |
| Green (500-570 nm) | 1.57 | 1.20 | 0.42 | 2.9 |
| Yellow (570-591 nm) | 2.57 | 1.98 | 0.69 | 4.9 |
| Orange (591-610 nm) | 1.79 | 1.46 | 0.50 | 3.5 |
| Red (610-700 nm) | 1.97 | 1.73 | 0.56 | 3.9 |
| TOTAL | 48.32 | 46.54 | 14.28 | 100.0% |

### INCORPORATION BY REFERENCE

All references cited in this specification, and their references, are incorporated by reference herein in their entirety where appropriate for teachings of additional or alternative details, features, and/or technical background.

### EQUIVALENTS

While the disclosure has been particularly shown and described with reference to particular embodiments, it will be appreciated that variations of the above-disclosed and other features and functions, or alternatives thereof, may be desirably combined into many other different systems or applications. Also, that various presently unforeseen or unanticipated alternatives, modifications, variations or improvements therein may be subsequently made by those skilled in the art which are also intended to be encompassed by the following embodiments.

1. A silicone-coated biophotonic material comprising: at least one biophotonic fiber component coated with silicone, wherein the at least one biophotonic fiber component is photo-stimulated upon exposure to light to emit fluorescence.

2. The silicone-coated biophotonic material according to claim 1, wherein the silicone-coated biophotonic material is a silicone-coated biophotonic membrane.

3. The silicone-coated biophotonic material according to claim 1, wherein the at least one biophotonic fiber component comprises biophotonic fibers.

4. The silicone-coated biophotonic material according to claim 1, wherein the biophotonic fibers are woven.

5. The silicone-coated biophotonic material according to claim 1, wherein the biophotonic fibers are non-woven.

6. The silicone-coated biophotonic material according to claim 1, wherein the biophotonic fibers comprise light-accepting molecules.

7. The silicone-coated biophotonic material according to claim 6, wherein the light-accepting molecules are xanthene dyes.

8. The silicone-coated biophotonic material according to claim 6, wherein the light-accepting molecules are Eosin Y.

9. The silicone-coated biophotonic material according to claim 6, wherein the light-accepting molecules are Eosin Y and Fluorescein.

10. The silicone-coated biophotonic material according to claim 1, wherein photo-stimulation of the at least one biophotonic fiber component causes the silicone-coated biophotonic material to emit fluorescence.

11. The silicone-coated biophotonic material according to claim 10, wherein the fluorescence emitted has a wavelength ranging from between 400 nm and about 700 nm.

12. The silicone-coated biophotonic material according to claim 1, wherein photo-stimulation of the at least one biophotonic fiber causes the silicone-coated biophotonic material to emit fluorescence in the yellow, orange and/or red regions.

13. The silicone-coated biophotonic material according to claim 1, wherein the biophotonic fibers are composed of nylon.

14. The silicone-coated biophotonic material according to claim 1, wherein interstices are present between fibers of the biophotonic fibers.

15. The silicone-coated biophotonic material according to claim 1, wherein the silicone coating has a thickness of between about 10 microns and about 100 microns.

16.-17. (canceled)

18. The silicone-coated biophotonic material according to claim 1, wherein the at least one biophotonic fiber component is a mesh.

19. The silicone-coated biophotonic material according to claim 1, wherein the at least one biophotonic fiber component is a flexible matrix.

20. The silicone-coated biophotonic material according to claim 1, wherein the at least one biophotonic fiber component is a patch.

21. The silicone-coated biophotonic material according to claim 1, wherein the photoactivated biophotonic fiber component emits in the purple, blue, green, yellow, orange and red wavelengths.

22.-23. (canceled)

24. A method for wound healing, the method comprising:
a) applying the silicone-coated biophotonic material according to claim 1; and
b) illuminating the silicone-coated biophotonic material with actinic light for a time sufficient to achieve photoactivation of the biophotonic fiber component.

25.-32. (canceled)
